(19) United States
(12) Patent Application Publication
(10) Pub. No.: US 2022/0372418 A1
(43) Pub. Date: Nov. 24, 2022

(54) Pumpless Microfluidic Devices and Uses Thereof

(71) Applicant: University of South Australia, Adelaide (AU)

(72) Inventors: Craig Ian Priest, Athelstone (AU); Edward Cheah, Mawson Lakes (AU); Ludivine Delon, Gatineau (CA); Azadeh Nilghaz, Scoresby (AU); Benjamin Thierry, Nailsworth (AU)

(21) Appl. No.: 17/774,078

(22) PCT Filed: Nov. 4, 2020
(86) PCT No.: PCT/AU2020/000127

§ 371 Date: May 3, 2022

(30) Foreign Application Priority Data
Nov. 4, 2019 (AU) 2019904151

(51) Int. Cl. C12M 3/06; C12M 1/34

(52) U.S. Cl. CPC C12M 23/16; C12M 41/44

(57) ABSTRACT

Provided is an apparatus for inducing and/or controlling flow of a fluid within a microchannel in a microfluidic device. The apparatus includes a fluid reservoir configured for holding a volume of fluid to be transported through said microfluidic channel and also configured for fluid connection to an inlet of said microfluidic channel. The apparatus also includes an evaporation reservoir configured for fluid connection to an outlet of said microfluidic channel. The evaporation reservoir includes at least one wetting, wicking or hydrophilic structure positioned at least partly within the reservoir. The wetting, wicking, or hydrophilic structure is capable of absorbing or conducting a fluid present in the microfluidic channel via wicking action or capillary force and maintaining a substantially constant volume of fluid in the evaporation reservoir. In use, evaporation of fluid at the outlet results in fluid being drawn from the fluid reservoir through the microfluidic channel to thereby create a flow of the fluid in the microfluidic channel.

### CROSS-REFERENCE TO RELATED APPLICATIONS

This application is the United States national phase of International Application No. PCT/AU2020/000127 filed Nov. 4, 2020, and claims priority to Australian Provisional Patent Application No. 2019904151 filed Nov. 4, 2019, the disclosures of which are hereby incorporated by reference in their entirety.

### BACKGROUND OF THE INVENTION

### Field of the Invention

The present disclosure relates generally to microfluidic devices and more specifically to fluid transportation in microfluidic devices.

### Description of Related Art

Microfluidic devices are being increasingly used in medical, biotechnology and analytical fields. For example, microfluidic devices are used to perform chemical analyses and microfluidic devices containing biomolecules are used in diagnosis and research.

In most, if not all, applications of microfluidic devices, a fluid is required to flow in one or more microfluidic channels. In these settings, fluid flow is typically induced by an external pump or pressure, such as peristaltic and syringe pumps. These microfluidic devices are therefore reliant on external equipment for their operation. Such requirements can limit the utility of these devices in certain settings, such as point-of-care settings, limited resource settings, and other environments with difficult or no access to necessary resources.

One particular application of microfluidic devices where fluid flow is important is cell culture. In cell culture applications, fluid flows are generally required to mimic a physiologically relevant situation, similar to flow of blood or other body fluids. However, despite ubiquitous use, the limitations of standard cell culture plasticware (i.e. cells grown in 2D on plastic substrates) are now well recognised. The organ-on-a-chip concept is the next generation of in vitro biological models but the inherent requirement for more complex culture conditions associated with organ-on-a-chip models is a major bottleneck in their implementation. To fulfil the tremendous potential of these advanced in vitro models, it is essential to develop the next generation of plasticware to allow the application of dynamic and 3D culture conditions without requiring complex instrumentation or equipment.

To address this need, there have been considerable efforts to date to develop organ-on-a-chip culture devices that do not require the use of external pumping. For example, Mimetas (J. H. Oortweg 19, 2333 CH Leiden, The Netherlands) has developed a range of organ-on-a-chip culture devices that use a “rocker” instead of a pump to achieve cell culture media flow within the devices. In these apparatus, a microfluidic device is placed on a laboratory rocker platform or table, whereby the microfluidic device is subjected to a reciprocating motion, such that levelling takes place between a pair of connected reservoirs in the microfluidic device thereby effecting a gravity-induced flow in the microfluidic channel as it is tilted to an angle on the rocker platform (see, for example, U.S. Pat. No. 8,748,180). This approach allows perfusion of fluid through microfluidic channels without a pump and tubing. However, there are significant disadvantages to these systems. In microfluidic applications such as cell culture, the samples are typically maintained under specific environmental conditions, e.g. incubation at a specific temperature, under humidified conditions and/or with determined and adjusted oxygen and/or carbon dioxide levels. However, the rockers are bulky and occupy quite substantial volumes in incubators. They also require an external source of energy to work, similar to pumps. In addition, there are significant costs associated with the purchase of such rockers. Finally, some of the present inventors have shown that shear stress initiated by a unidirectional flow plays a key role in regulating the structure and functions of epithelial monolayers and that fine-tuning of the shear-stress is essential. However, in the rocking system, the shear is brought about by a bidirectional and oscillating flow going forward and backward which is not what happens in vivo and is difficult to define.

There is a significant need for apparatus and methods to induce and/or control flow of fluids in microchannels without the requirement to have substantial external equipment such as pumps, vacuums, and other pressure sources.

### SUMMARY OF THE INVENTION

In a first aspect, provided herein is an apparatus for inducing and/or controlling flow of a fluid within a microfluidic channel in a microfluidic device, the apparatus comprising:

a fluid reservoir configured for holding a volume of fluid to be transported through said microfluidic channel and also configured for fluid connection to an inlet of said microfluidic channel;

an evaporation reservoir configured for fluid connection to an outlet of said microfluidic channel, the evaporation reservoir comprising at least one wetting, wicking or hydrophilic structure positioned at least partly within the reservoir, the wetting, wicking, or hydrophilic structure capable of absorbing or conducting a fluid present in the microfluidic channel via wicking action or capillary force and maintaining a substantially constant volume of fluid in the evaporation reservoir;

wherein controlled evaporation of fluid at the outlet results in fluid being drawn from the fluid reservoir through the microfluidic channel to thereby create a flow of the fluid in the microfluidic channel.

Advantageously, the apparatus of the first aspect does not require the use of any power source or substantial external equipment in order to effect, maintain and/or control fluid flow in the microfluidic channel. It allows subjecting single, but also a multitude of microfluidic devices, to a standardised movement and permits incubation under desired conditions.

In certain embodiments of the first aspect, the wetting, wicking, or hydrophilic structure comprises a plurality of wicking strands. Each wicking strand may be composed of a single strand of a wicking material. The wicking material may be a hydrophilic polymer material. The hydrophilic polymer material may comprise a polymer coated with a hydrophilic coating.

In a second aspect, provided herein is an arrangement comprising a plurality of the apparatuses of the first aspect.

In a third aspect, provided herein is a method for providing fluid flow to one or more microfluidic devices, the method comprising:

providing an apparatus of the first aspect;

adding a fluid to the fluid reservoir; and

maintaining the apparatus under conditions that result in evaporation of the fluid at the outlet to thereby create a flow of the fluid in the microfluidic channel.

### BRIEF DESCRIPTION OF DRAWINGS

Embodiments of the present disclosure will be discussed with reference to the accompanying drawings wherein:

FIG. 1 is a schematic representation (left) and a photograph (right) of a serpentine microfluidic chamber according to embodiments of the invention. The flow rate of Caco-2 cells in the chamber was 0.15 μL/min and the flow rate of A549 cells in the chamber was 1.6 μL/min;

FIG. 2 is a graphical representation (left) and a photograph (right) of a Hele-Shaw microfluidic chamber according to embodiments of the invention. The flow rate of Caco-2 cells in the chamber was 0.22 μL/min and the flow rate of A549 cells in the chamber was 3.0 μL/min;

FIG. 3 shows SEM microphotographs of super-hydrophilic threads used in microfluidic chambers according to embodiments of the invention;

FIG. 4 is a plot showing the capillary rise measurements of the threads shown in FIG. 3. Different widths of thread were tested, namely 0.5 mm (∘), 1.0 mm (□), and 1.5 mm (Δ). Threads of 10 cm length were dipped into a dye and level (wicking height) of the dye was measured over time for 2 minutes. This shows the absorption capacity of the fibre used;

FIG. 5 is a schematic representation showing details of the fabrication of a serpentine microfluidic chamber according to embodiments of the invention;

FIGS. 6A-C are a real-time visualisation of nanoparticles moving within the microfluidic channel driven by a thread, video recorded using fluorescence microscope (A): 5 mm thread length, (B): 13 mm thread length, and (C): 20 mm thread length. The distance that 200 nm nanoparticles (FluoSpheres) travelled along the channel over time reveals the velocity of nanoparticles. The flow rate is calculated using the formula: Flow rate (μL/min)=Velocity (μm/min)×Area of the Channel (μm²);

FIG. 7 is a plot showing the flow rate calculations. Based on the above formula, the flow rate was measured using the serpentine chip and testing different length of the thread (this length refers to the part of the thread standing outside of the outlet of the chip);

FIG. 8 is a plot of thread length and shear stress for Hele-Shaw microfluidic devices with shear stresses applied to cells (Caco-2 intestinal cells) along the channel as demonstrated in Biomaterials; Delon et al., 2019, Systematic investigation of the effect of the fluid shear stress on Caco-2 cells towards the optimization of epithelial organ-on-chip models;

FIG. 9 shows Caco-2 cells in a Hele-Shaw device. Caco-2 cells (enterocyte phenotype) can grow normally and achieve confluence in the Hele-Shaw device using the thread at optimized flow rate;

FIG. 10 shows Caco-2 cells in a Hele-Shaw device. Caco-2 cell monolayer displayed a similar differentiation than previously demonstrated, here shown with F-actin expression and height of the monolayer across the different sections;

FIG. 11 shows Caco-2 cells in Hele-Shaw device. Caco-2 cell monolayer displayed a similar differentiation than previously demonstrated, here shown with the number of active mitochondria across the different sections;

FIG. 12 shows Caco-2 cells and A549 cells in serpentine devices. Caco-2 cells grown under 0.02 dyn/cm². Thread: 2 mm length. A549 cells grown under 0.4 dyn/cm². Thread: 2 cm length. Typically, epithelial cells experience much lower fluid shear stresses than endothelial cells but the intestinal cells and lung cells also have very different conditions. Intestinal cells (like Caco-2 cell line) are known to proliferate and differentiate well under low shear (˜0.005-0.025 dyn/cm²), and A549 cells have been shown to undergo epithelia-mesenchymal transition with more moderate shear stress (1.4 to 2 dyn/cm² or more). See: Flitney, E. W., et al., 2009. Insights into the mechanical properties of epithelial cells: the effects of shear stress on the assembly and remodelling of keratin intermediate filaments. The FASEB Journal, 23(7), pp. 2110-2119; and Rizvi, I., et al, 2013. Flow induces epithelial-mesenchymal transition, cellular heterogeneity and biomarker modulation in 3D ovarian cancer nodules. Proceedings of the National Academy of Sciences, 110(22), pp. E1974-E1983;

FIG. 13 is a photograph of a multi-channel microfluidic device in accordance with embodiments of the invention having two microfluidic channels separated by a gel channel and suitable for co-culture of two types of cells;

FIG. 14 is a photograph of a multilayer microfluidic device in accordance with embodiments of the invention having two microfluidic channels, one on top of the other, and separated by a porous membrane and suitable for co-culture of two types of cells;

FIG. 15 shows SEM microphotographs of A549 cells and lung fibroblast cells cultured (Day 5) in a co-culture microfluidic device in accordance with embodiments of the invention; and

FIG. 16 shows SEM microphotographs of Caco2 cells and intestinal fibroblast cells cultured (Day 5) in a co-culture microfluidic device in accordance with embodiments of the invention.

### DESCRIPTION OF THE INVENTION

Disclosed herein is an apparatus for inducing and/or controlling flow of a fluid within a microfluidic channel in a microfluidic device. The apparatus comprises a fluid reservoir configured for holding a volume of fluid to be transported through said microfluidic channel and also configured for fluid connection to an inlet of said microfluidic channel. The apparatus also comprises an evaporation reservoir configured for fluid connection to an outlet of said microfluidic channel. The outlet comprises at least one wetting, wicking or hydrophilic structure positioned at least partly within the reservoir. The wetting, wicking, or hydrophilic structure is capable of absorbing or conducting a fluid present in the microfluidic channel via wicking action or capillary force and maintaining a substantially constant volume of fluid in the evaporation reservoir. In use, evaporation of fluid at the outlet results in fluid being drawn from the fluid reservoir through the microfluidic channel to thereby create a flow of the fluid in the microfluidic channel.

The apparatus described herein has a wide number of uses in chemistry, biology and biomimetic applications. For example, the apparatus may be utilised in processes that exploit microfluidic technology and require fluid flow through a channel, such as chemical synthesis, concentration of analytes by evaporation, filtration, purification processes, biomimetic processes, biological processes, and analytical processes. For ease of description and understanding, we will now refer to illustrated embodiments that are suitable for use in microfluidic organ-on-a-chip devices. As an organ-on-a-chip device, the present inventors have demonstrated that the apparatus described herein can be used, for example as a lung-on-a-chip or an intestine-on-a-chip.

The apparatus described herein has potential to be widely used in research institutes and biotechnology companies active in the fields of pharmacy, virology, parasitology, microbiology and those using primary cells and organoids from patients. In addition, pumpless devices eliminate potential risks associated with the use of tubing and/or electrical apparatus (e.g. pump within an incubator), which is appealing for research involving biohazards.

As mentioned, the apparatus described herein is particularly useful for microfluidic organ-on-a-chip applications. Microfluidic organ-on-a-chip devices are rapidly being developed towards eliminating the shortcomings of standard static in vitro models and better addressing both basic and translational research questions. The global organ-on-a-chip market was valued at $5 million in 2016, and is projected to reach at least $170 million by 2023, growing at a CAGR of 63.2% from 2017 to 2023.

A key feature of microfluidic organ-on-a-chip devices is that they provide a dynamic culture environment that significantly impacts the phenotype and functions of the cultured cells.

At least some of the present inventors have recently demonstrated that fine control of fluid shear stress is essential to realise the potential of organ-on-a-chip devices. However, the associated inherent requirement for precise fluidic conditions and therefore the need for precise pumps is a bottle-neck that limits the implementations of these models in laboratories with limited microfluidic experience and facilities.

The pumpless apparatus described herein is equipment-free and could be readily employed by non-specialist end-users for preparing organ-on-a-chip based on cells lines or organoids. This makes microfluidic organ-on-a-chip more accessible and easier to manipulate due to the absence of tubing and pumps or rockers in cell incubators.

The apparatus described herein is used to induce and/or control laminar flow of a Newtonian fluid within a microfluidic channel in a microfluidic device. Fluid flow is induced or controlled by the application of hydrophilic materials such as fibres, which provide a driving force to achieve continuous flow of a fluid, such as a culture medium, over 24 hours without the requirement for either a pump or external power source.

As used herein, the term “microfluidic”, and variants thereof, means that the chip, device, apparatus, substrate or related apparatus contains fluid control features that have at least one dimension that is sub-millimetre and, typically less than 1000 μm, and greater than 1 μm. Furthermore, the terms “microfluidic channel”, “microchannel”, and variants thereof, means a channel having at least one dimension that is sub-millimetre and, typically less than 1000 μm, and greater than 1 μm.

The microfluidic channel is part of a microfluidic device comprising a solid substrate comprising the microfluidic channel and any other microfluidic features as required. Materials suitable for the manufacture of solid substrates for microfluidic elements are known in the art and may be chosen based on considerations such as cost, inertness or reactivity toward fluids and other materials that will be in contact with the substrates, etc. Some examples of suitable materials include glass, quartz, metal (e.g. stainless steel), ceramic, silicon, and polymers. Suitable polymeric materials include polydimethylsiloxane (PDMS), polytetrafluoroethylene (PTFE), other perfluoropolyether (PFPE) based elastomers, polymethylmethacrylate (PMMA), silicone, and the like. The solid substrates in the illustrated embodiments are rectangular in plan view but it is envisaged that they can be other shapes in plan view, such as square, circular, etc.

Furthermore, the solid substrate can be in the form of a chip. If there is more than one solid substrate, the two or more substrates may be connected to one another in series or parallel using suitable tubing and connectors, as is known in the art. For example, a through-hole can be used to connect an upper solid substrate and a lower solid substrate.

The microfluidic channel (and/or any other microfluidic features) of the microfluidic device can be formed in the solid substrate using any of the techniques for forming fluid microchannel networks that are known in the art. For example, the substrates can be fabricated using standard photolithographic and etching procedures including soft lithography techniques (e.g. see Shi J., et al., Applied Physics Letters 91, 153114 (2007); Chen Q., et al., Journal of Microelectromechanical Systems, 16, 1 193 (2007); or Duffy et al., Rapid Prototyping of Microfluidic Systems in Poly(dimethylsiloxane), Anal. Chem., 70 (23), 4974-4984 (1998)), such as near-field phase shift lithography, microtransfer molding, solvent-assisted microcontact molding, microcontact printing, and other lithographic microfabrication techniques employed in the semiconductor industry. Direct machining or forming techniques may also be used as suited to the particular device. Such techniques may include hot embossing, cold stamping, injection molding, direct mechanical milling, laser etching, chemical etching, reactive ion etching, physical and chemical vapour deposition, and plasma sputtering. The particular methods used will depend on the function of the particular microfluidic network, the materials used as well as ease and economy of production.

The microfluidic devices disclosed herein can incorporate such valves and other microfluidic features, flow channels, dead channels, reservoirs, as required.

The microfluidic device can be fabricated as shown in FIG. 5. A PDMS solid substrate 10 having a serpentine microfluidic channel 12 was cast. An 8 mm diameter fluid reservoir 14 was then punched at the inlet 16 to the microfluidic channel 12. A 3 mm diameter outlet (evaporation reservoir) 18 was punched at the outlet 20 of the microfluidic channel 12 and the evaporation reservoir 18 was further punched with a 1.5 mm hole 22. The PDMS solid substrate 10 was plasma bonded to a super thin glass cover slip. The PDMS element was then used to insert a wicking strand 24 so that it didn't touch the bottom of the evaporation reservoir 18. A lid 26 was placed at the fluid reservoir 14 to limit evaporation of fluid contained therein.

The fluid reservoir 14 is configured for holding a volume of fluid to be transported through the microfluidic channel 12 and also configured for fluid connection to the inlet 16 of the microfluidic channel 12. The fluid reservoir 14 may be part of the microfluidic device or it may be separate from the microfluidic device but be in the fluid connection with the inlet 16 of the microfluidic channel 12 on the microfluidic device.

The evaporation reservoir 18 is configured for fluid connection to the outlet 20 of the microfluidic channel 12. The evaporation reservoir 18 may be part of the microfluidic device or it may be separate from the microfluidic device but be in the fluid connection with the outlet 20 of the microfluidic channel 12 on the microfluidic device.

The evaporation reservoir 18 comprises at least one wetting, wicking or hydrophilic structure 24. In certain embodiments, the wetting, wicking, or hydrophilic structure comprises one or more wicking strands. Each wicking strand may be composed of a single strand of a wicking material. The wicking material may be a hydrophilic polymer material. The hydrophilic polymer material may comprise a polymer coated with a hydrophilic coating.

It will be appreciated that the wetting, wicking or hydrophilic structure 24 provides a relatively large and well controlled surface area from which evaporation of a fluid can occur. The thickness and the length of the wicking strands in the wetting, wicking or hydrophilic structure 24 play an important role in generating a tuneable flow rate. This is particularly useful for different organ-on-a-chip models because it enables the application of well-defined fluidic shear forces and as a result the tuning of the structure and phenotype of the cultured cells.

The present inventors have observed that flow rates of fluid in the microfluidic channel 12 can be accurately controlled based on characteristics of the wetting, wicking or hydrophilic structure 24. Temperature, humidity, and air circulation speed all affect evaporation and, therefore, control of one or more of these can also be used to control the rate of evaporation of fluid at the outlet 20 via the evaporation reservoir 18 and hence the flow rate of fluid in the microfluidic channel 12. Surface tension is also important. It will be appreciated that the rate of evaporation can be kept constant in a cell culture incubator and, therefore, the apparatus described herein is particularly useful for biological or biomimetic applications that are carried out in an incubator, such as organs-on-a-chips.

By way of example, flow rates up to 10 μl/min can be achieved using the apparatus described herein. The flow rate, and therefore the fluid shear stress experienced by the cells, for organ-on-a-chip devices should be carefully adjusted to obtain the desirable phenotype and functions for specific cells. For example, flow rates in the range of from about 0.05 μl/min to about 1 μl/min can be used for the culture of epithelial cells. On the other hand, cell types such as endothelial cells and stromal cells may be exposed to a higher flow rate of up to 10 μL/min for example.

Advantageously, the present inventors have observed that flow rates of fluid in the microfluidic channel 12 can provide a suitable range of fluid shear stress conditions for cells.

In certain embodiments, the wicking strand is a super hydrophilic fibre (wet and dry tensile strength (45 gsm fabric)), Spunlace (70% Rayon, 30% Polyester) from Ebos Healthcare Australia.

In certain other embodiments, the wetting, wicking or hydrophilic structure 24 comprises a pillar array. For example, a 6×6 mm pillar array may comprise a plurality of pillars. The fluid volume in the pillar array may be <1 μL. Examples of suitable pillar arrays are disclosed in, for example, published International (PCT) patent application WO 2016/090407 A1.

The wetting, wicking, or hydrophilic structure 24 is capable of absorbing or conducting a fluid present in the microfluidic channel 12 via wicking action or capillary force and maintaining a substantially constant volume of fluid in the evaporation reservoir 18.

The wetting, wicking or hydrophilic structure 24 is positioned at least partly within the evaporation reservoir 18 such that one end of the structure 24 is in contact with fluid at the outlet 20 of the microfluidic channel 12. In this way, the hydrophilic structure 24 wicks the fluid by capillary action. The other end of the structure 24 extends from the evaporation reservoir 18 and is exposed to allow evaporation of fluid present on the structure 24. In this way, as the fluid evaporates from the structure 24 more fluid is drawn into the structure 24. This, in turn, results in fluid flowing from the fluid reservoir 14 along the microfluidic channel 12. Thus, evaporation of fluid at the outlet 20 via the evaporation reservoir 18 results in fluid being drawn from the fluid reservoir 14 through the microfluidic channel 12 to thereby create a flow of the fluid in the microfluidic channel 12.

Advantageously, the wicking strand 24 is loosely held in the hole 22 and is able to move longitudinally in the hole 22. The wicking strand 24 may be at least partially buoyant in the fluid such that when fluid is present in the evaporation reservoir 18 the end of the wicking strand 24 is spaced from the outlet 20 of the microfluidic channel 12 and the fluid is able to flow out of the microfluidic channel 12. However, when the fluid level in the evaporation reservoir 18 drops, such as may happen when the fluid in the fluid reservoir 14 runs out, the wicking strand 24 drops in the hole 22 and can reach a point where the end of the wicking strand 24 at least partially blocks the outlet 20 of the microfluidic channel 12 to thereby minimise or prevent further flow of fluid out of the microfluidic channel 12. This effectively provides a valve that minimises or prevents fluid from completely running out of the microfluidic channel 12. Thus, if the fluid in the fluid reservoir 14 runs dry, then there may still be fluid remaining in the microfluidic channel 12.

In another embodiment, an end of the wicking strand 24 that is adjacent the evaporation reservoir 18 is spaced from the bottom of the reservoir 14 by a predefined distance. In this embodiment, if the fluid reservoir 14 runs out of fluid, the outlet reservoir 20 will have fluid remaining in it because the wicking strand 24 does not touch the bottom of the evaporation reservoir 18. In this way, the microfluidic channel 12 does not empty because the wicking strand 24 stops contacting the fluid once the level drops below the “safety level” as determined by the predetermined distance.

The fluid can be any suitable Newtonian liquid, fluid or medium such as any aqueous solution including, but not limited to, water, buffered solution, growth medium, etc. The fluid may contain organic solvents as required.

In certain embodiments, the apparatus may further comprise a dead volume reservoir positioned between the microfluidic channel 12 and the evaporation reservoir 18. The dead volume reservoir provides further fluid volume capacity for the apparatus which then means that the apparatus can be run for long periods of time without losing fluid volume in the microfluidic channel 12.

Advantageously, the apparatus described herein does not require the use of any substantial external equipment in order to effect, maintain and/or control fluid flow in the microfluidic channel 12. It allows subjecting single, but also a multitude of microfluidic devices, to a standardised movement and permits incubation under desired conditions.

In certain embodiments, the microfluidic device comprises more than one microfluidic channel. Each microfluidic channel in such a “multichannel” device is driven by separate wetting, wicking or hydrophilic structures 24. Different cells can be cultured in each of the different microfluidic channels (which may be used to induce different flow rates adjusted for the cell type in each channel). The cells can be either separated by a porous membrane (with pores<cell diameters) in a “multi-layer” configuration (i.e. vertical) or in a multi-channel configuration where the channels are separated by PDMS features that contain the flow within the separate channels.

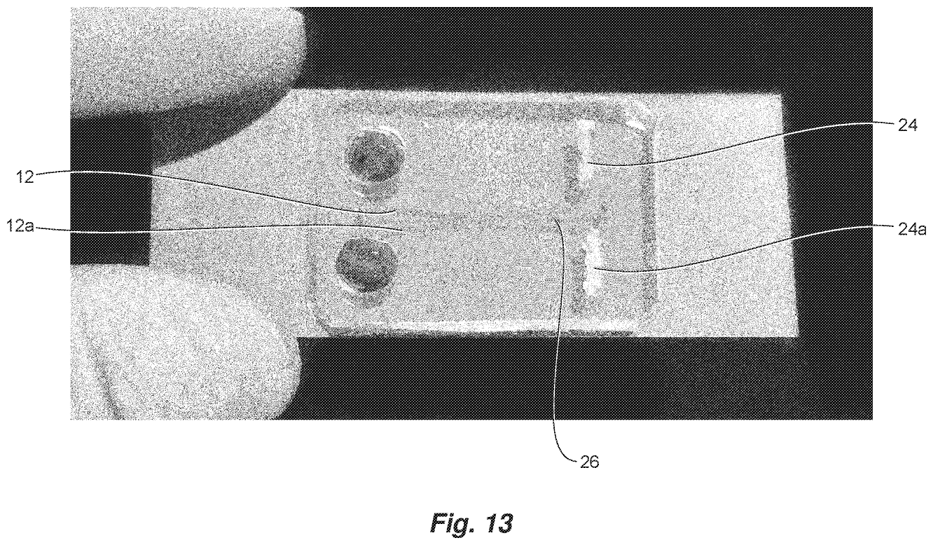

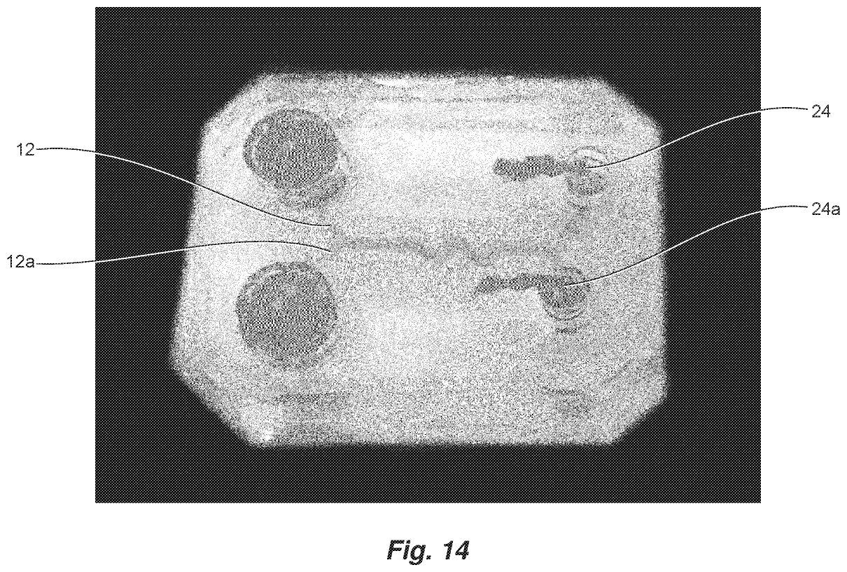

In certain embodiments, the microfluidic device can be used for the co-culture of cells. The microfluidic device may have one of two typical configurations for co-culture, as shown in FIGS. 13 and 14. Shown in FIG. 13 is a multi-channel configuration wherein two microfluidic channels 12 and 12a are separated by a channel filled with gel 26 and two types of cells are cultured on either side of the gel channel 26. Each respective channel 12 and 12a is perfused with media driven by a respective wetting, wicking or hydrophilic structures 24 and 24a. This allows independent control of the flow rate in these two channels.

Shown in FIG. 14 is a multi-layer configuration suitable for co-culture of cells. In this configuration, microfluidic channels 12 and 12a are on “top of each other” and separated by a semi-porous membrane that allows exchange of media and biomolecules. The two microfluidic channels 12 and 12a are independently perfused with media driven by a respective wetting, wicking or hydrophilic structures 24 and 24a. This again allows independent control of the flow rate in these two channels 12 and 12a.

Also disclosed herein is an arrangement comprising a plurality of the apparatuses of the first aspect.

Also disclosed herein is a method for providing fluid flow to one or more microfluidic devices. The method comprises providing an apparatus as described herein, adding a fluid to the fluid reservoir 14; and maintaining the apparatus under conditions that result in evaporation of the fluid at the outlet 20 to thereby create a flow of the fluid in the microfluidic channel 12.

Advantageously, the microfluidic devices described herein may be relatively optically transparent and this means that cell growth, reactions and the like can be readily monitored using a variety of different detection systems at essentially any location on the microfluidic device.

### Examples

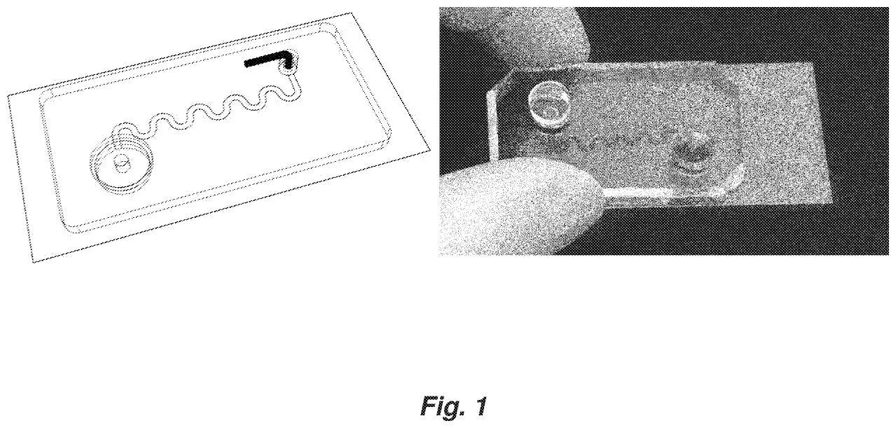

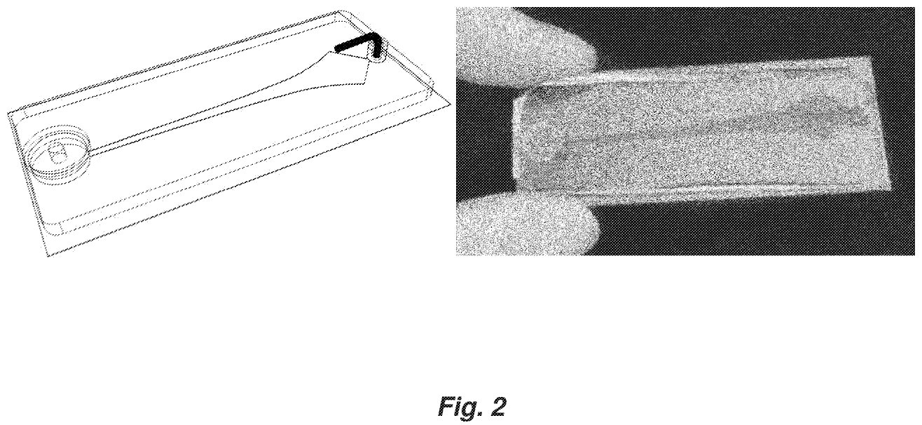

Several organ-on-chip configurations were successfully tested, including devices suitable for cellular monoculture and devices suitable for co-culture. Monoculture devices consisted of a single fluidic channel made of PDMS, bonded to a glass cover slip (Proscitech, G418, No 1). Both constant width channels (FIG. 1) and channels with a Hele-Shaw geometry (FIG. 2) were tested. Master moulds were fabricated using standard soft lithography techniques. Briefly, SU-8 photoresist 50 was spun using a Karl Suss Delta 80 spin coater (Suss MicroTec, Germany) to produce a 150 μm layer (2000 rpm spin) on a silicon wafer. The microfluidic channel design was created with a Dilase 650 mask writer (Kloe, France), followed by the development and thermal hardening of the SU-8. PDMS (10:1, w/w) was cast onto the silicon micropatterned master mould and cured for 2 hours at 70° C. A 10 mm thick PDMS layer was cut and peeled off, then punched to create 6-8 mm hole for the inlet reservoir (note that this can be made bigger on a thicker PDMS layer to make a larger reservoir if required), while a 3 mm hole was punched at the outlet and itself punched with a 1-1.5 mm hole. This small PDMS element of 3 mm diameter punched with a hole of 1-1.5 mm is later used to insert the thread such as it doesn't touch the bottom. The PDMS layer was then oxygen plasma treated, bound to a glass cover slip and maintained for 10 min at 65° C. to produce permanent bonding between the glass coverslip and the PDMS block defining the microfluidic channel.

Human intestinal epithelial Caco-2 cells (Caco-2 BBE human colorectal carcinoma line) (Peterson M D, and Mooseker, M, J Cell Sci. 1992; 102:581-600) were cultured in 75 cm² tissue culture flasks in Dulbecco's Modified Eagle Medium (DMEM, Sigma Aldrich, Australia) F-12 Ham supplemented with 10% foetal bovine serum (Life Technologies), 1% L-glutamine (Sigma Aldrich) and 1% streptomycin/penicillin (Sigma Aldrich) at 37° C. and at 5% CO₂ levels in a humidifying incubator. Human lung epithelial A549 cells (ATCC® CCL-185™ human lung carcinoma line) were cultured in 75 cm² tissue culture flasks in DMEM F12-K mixture (Gibco, ThermoFisher, Australia) supplemented with 10% FBS, 1% L-glutamine and 1% streptomycin/penicillin at 37° C. and at 5% CO₂ levels in a humidifying incubator. Before seeding the devices, the channels were treated with 70% (v/v) ethanol and then washed with cold phosphate buffered saline (PBS, Ca′ and Mg′ free, pH=7.4, Sigma Aldrich), each for 30 min at room temperature (RT) in a biosafety hood. Each device was then transferred to a sterile Petri dish. A cold 1% (v/v) Matrigel Basement Membrane Matrix solution (Corning, BD Biosciences, Tewksbury, Mass., USA) in serum-free culture medium was then perfused and left to incubate in the device for 1 hour at 37° C. under static conditions to coat the glass cover slip surface. Cells were harvested with 0.25% trypsin/EDTA (Sigma Aldrich) and pipetted inside the device at a concentration of 2×10⁵ cells/cm² and left to adhere to the Matrigel coated glass for at least 1 hour. The total area of the surface area for the constant-width channel was 0.35 cm² and the total volume was 5.25 μL. The total area of the Hele-Shaw channel was 2.552 cm² and the total volume was 38.28 Following cell attachment, a thread was inserted in the outlet as described earlier. The cell media flow rate post attachment for the constant-width channel was set at 0.15 μL/min, and for the Hele-Shaw chamber at 0.22 μL/min for Caco-2 cells. This corresponded in the device used here to an applied shear stress of 0.02 dyn/cm² in the constant-width channels. The Caco-2 cells were grown for 5 days to form a confluent and fully-differentiated monolayer, as previously described (Pocock K, Delon, L., Bala, V., Rao, S., Priest, C., Prestidge, C. A., & Thierry, B. ACS Biomaterials Science & Engineering. 2017; 3:951-9.). The cell density was monitored daily using bright field microscopy on an inverted Eclipse Ti-E Nikon microscope (Nikon, Japan). Monolayers were also imaged using Hoechst for live nuclei staining. A549 cells were cultured under higher flow rates corresponding to higher shear stress values (e.g. 0.075 dyn/cm² in the constant-width channel). Indeed, intestinal cells (like Caco-2 cell line) are known to proliferate and differentiate well under low shear in such device (˜0.005-0.025 dyn/cm²), while lung cells (like A549 cell line) have been shown to undergo epithelia-mesenchymal transition with moderate shear stress (up to 2 dyn/cm² or more).

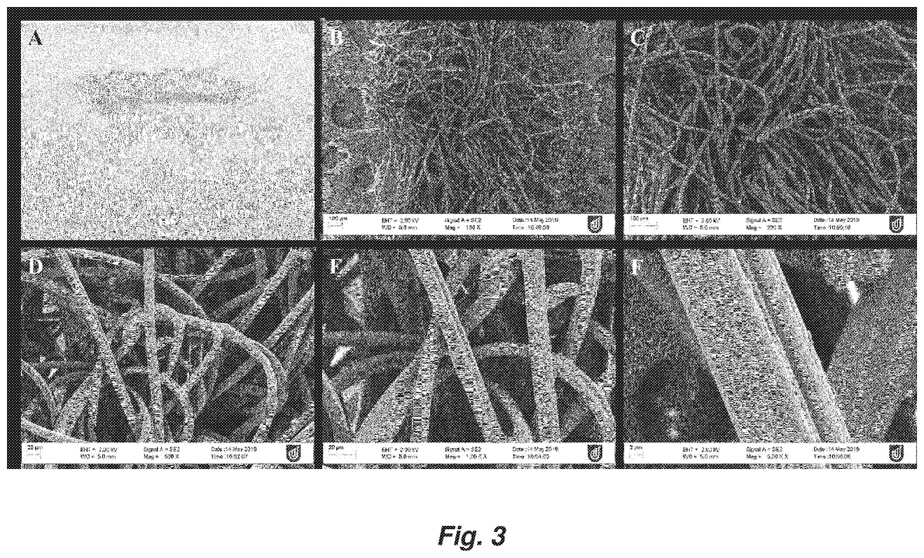

A super hydrophilic fibre (Wet and dry tensile strength (45 gsm fabric), Spunlace (70% Rayon, 30% Polyester) from Ebos Healthcare Australia) was used. The fiber was characterized using a benchtop scanning electron microscope (SEM, JEOL Neoscope (JCM-5000)) with an acceleration of 2 kV (FIG. 3).

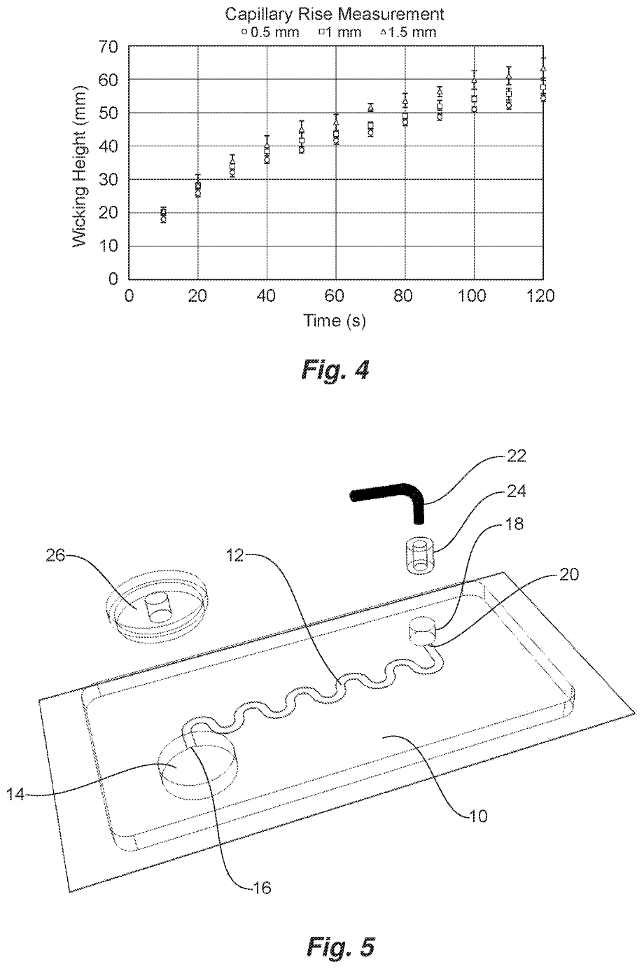

To determine the capillary properties of the super hydrophilic thread, three different widths of thread were tested (0.5, 1 and 1.5 mm). Briefly, 10 cm thread lengths were dipped into a dye solution and the levels (wicking heights) of the dye was measured over time for 2 minutes (FIG. 4).

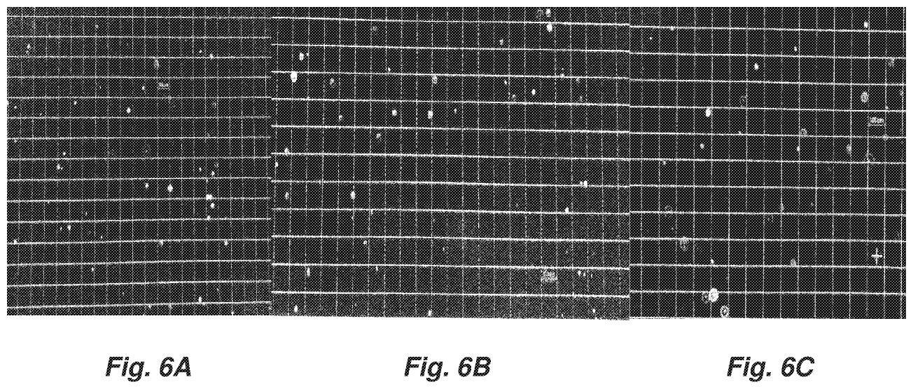

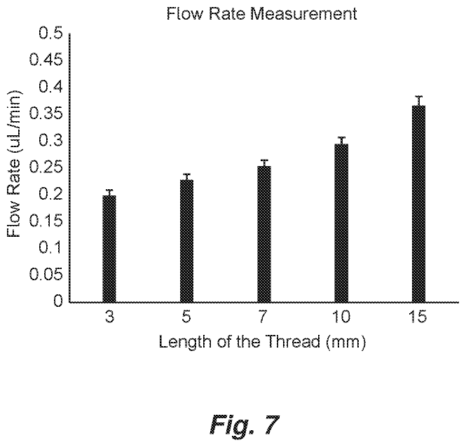

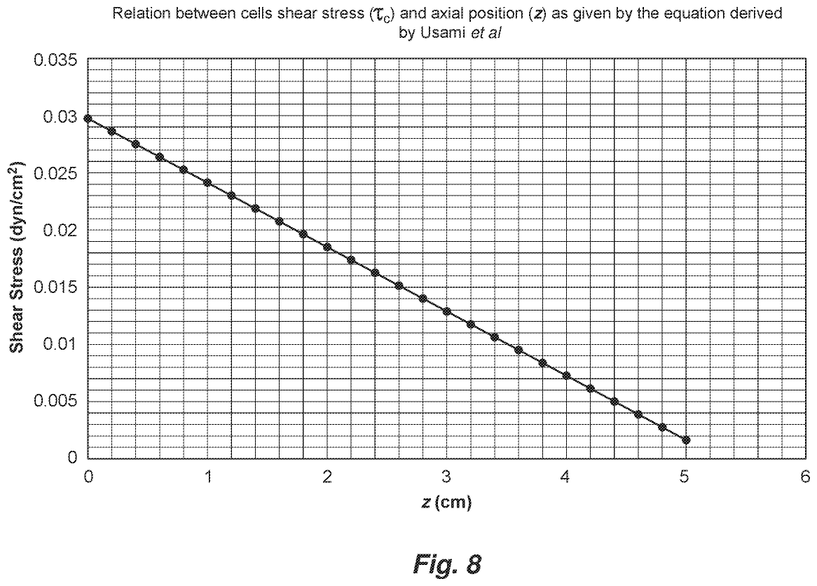

The flow behaviour induced within the microfluidic channel by a thread of specific characteristics was verified experimentally by measuring the velocity of highly fluorescent nanoparticles. The movement of 200 nm polystyrene beads (FluoSpheres, ThermoFisher) within a constant-width channel was monitored with an inverted Eclipse Ti-E Nikon microscope (Nikon, Japan) equipped with an ANDOR zyla 5.5 camera (Andor Technology Ltd, Belfast Northern Ireland) using 20× objective. The NIS-Elements BR (Nikon, Japan) software and Icecream App were used for screen recording. The average flow rate was approximated using the equation Q_{fluid}=V_{particles}×A_{channel} with Q_{fluid} the flow rate (μL/min); A_{channel}, the cross-sectional area of the channel (0.15 mm² in this study) and V_{particles} the averaged velocities of particles in the middle of the channel (in mm/min). Each experiment was repeated at least three times (FIGS. 6A-C and 7).

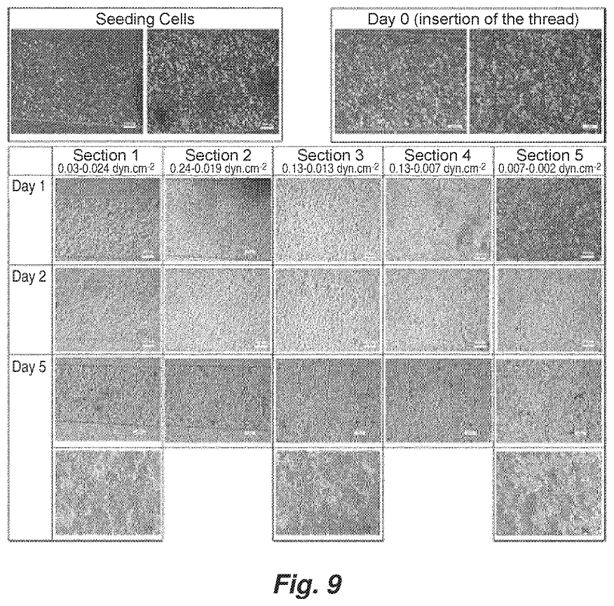

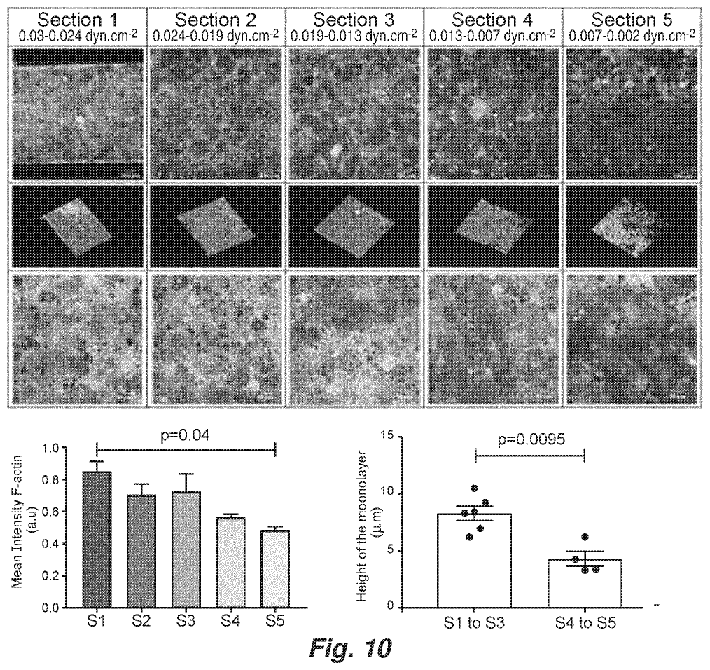

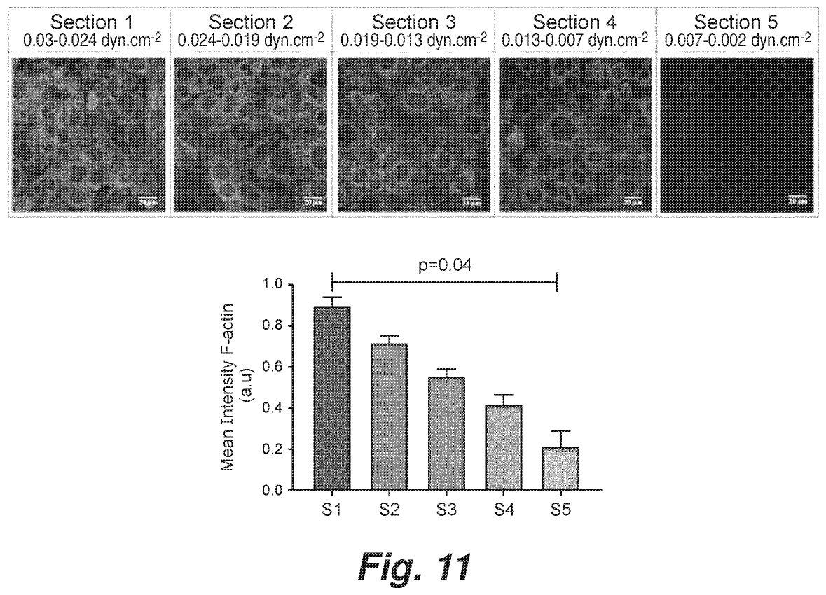

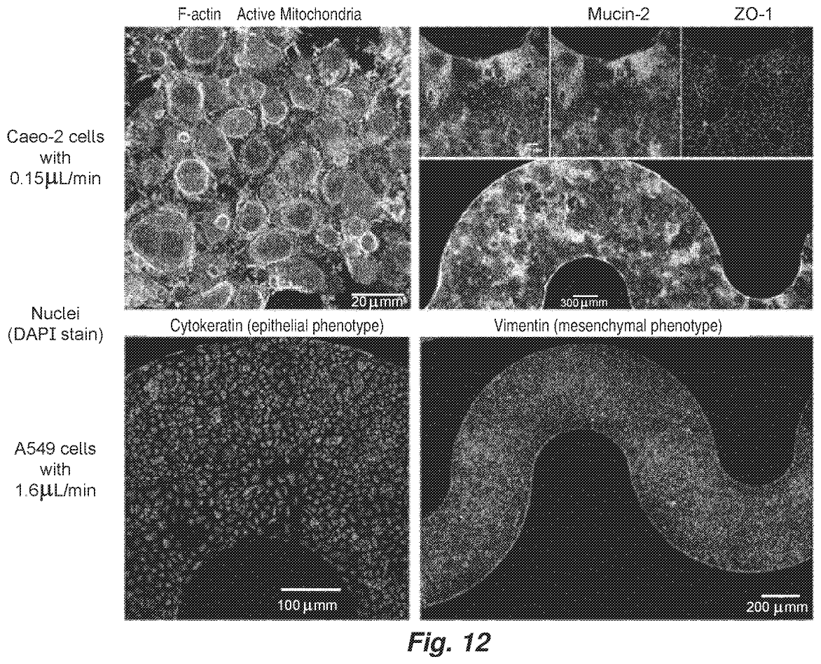

The cells cultured within the device under flow driven by a thread were regularly observed with bright field microscopy (FIG. 9), confirming the formation of dense monolayers within a few days of culture for both Caco-2 cells and A549 cells Immunostaining was also used to characterize the monolayers, for example for expression of f-actin (FIG. 10) and mitochondrial activities (FIGS. 11 and 12) of cells exposed to different shear stresses.

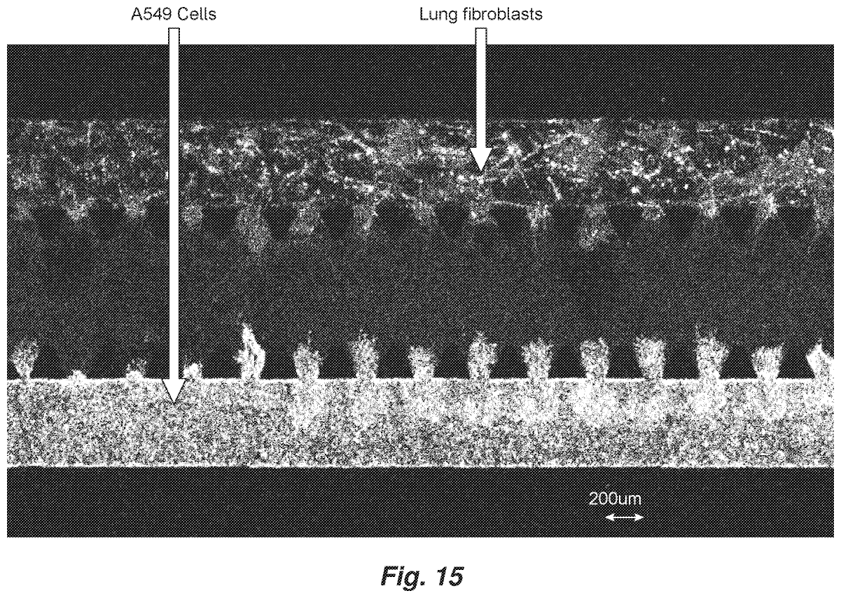

In a further experiment, A549 cells and lung fibroblast cells were successfully co-cultured (using the conditions described above) in a dual channel microfluidic device of the type shown in FIG. 13 or FIG. 14. The results are shown in FIG. 15.

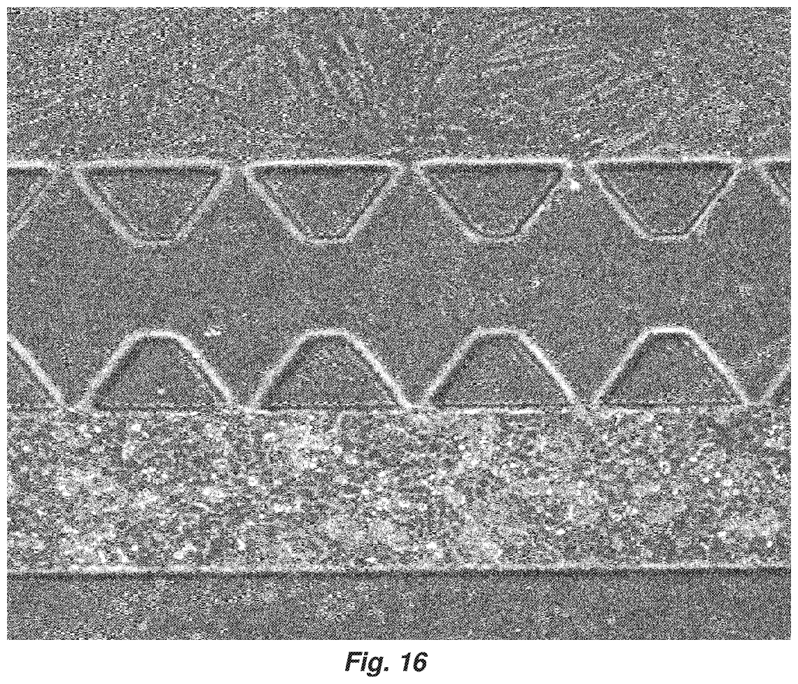

In still a further experiment, Caco-2 cells and intestinal fibroblast cells were successfully co-cultured (using the conditions described above) in a dual channel microfluidic device of the type shown in FIG. 13 or FIG. 14. The results are shown in FIG. 16.

Throughout the specification and the claims that follow, unless the context requires otherwise, the words “comprise” and “include” and variations such as “comprising” and “including” will be understood to imply the inclusion of a stated integer or group of integers, but not the exclusion of any other integer or group of integers.

The reference to any prior art in this specification is not, and should not be taken as, an acknowledgement of any form of suggestion that such prior art forms part of the common general knowledge.

It will be appreciated by those skilled in the art that the invention is not restricted in its use to the particular application described. Neither is the present invention restricted in its preferred embodiment with regard to the particular elements and/or features described or depicted herein. It will be appreciated that the invention is not limited to the embodiment or embodiments disclosed, but is capable of numerous rearrangements, modifications and substitutions without departing from the scope of the invention as set forth and defined by the following claims.

1. An apparatus for inducing and/or controlling flow of a fluid within a microfluidic channel in a microfluidic device, the apparatus comprising:
a fluid reservoir configured for holding a volume of fluid to be transported through said microfluidic channel and also configured for fluid connection to an inlet of said microfluidic channel; and
an evaporation reservoir configured for fluid connection to an outlet of said microfluidic channel, the evaporation reservoir comprising at least one wetting, wicking or hydrophilic structure positioned at least partly within the reservoir, the wetting, wicking, or hydrophilic structure capable of absorbing or conducting a fluid present in the microfluidic channel via wicking action or capillary force and maintaining a substantially constant volume of fluid in the evaporation reservoir;
wherein evaporation of fluid at the outlet results in fluid being drawn from the fluid reservoir through the microfluidic channel to thereby create a flow of the fluid in the microfluidic channel.

2. The apparatus according to claim 1, wherein the wetting, wicking, or hydrophilic structure comprises a plurality of wicking strands.

3. The apparatus according to claim 2, wherein each wicking strand is composed of a single strand of a wicking material.

4. The apparatus according to claim 3, wherein each wicking strand comprises a hydrophilic polymer material.

5. The apparatus according to claim 4, wherein each wicking strand comprises 70% Rayon and 30% polyester.

6. The apparatus according to claim 5, wherein an end of each wicking strand that is adjacent a bottom of the evaporation reservoir is spaced from the bottom of the reservoir by a predefined distance thereby preventing the microfluidic channel from emptying when the fluid reservoir empties because each wicking strand stops contacting the fluid once the fluid level in the evaporation reservoir drops below a level as determined by the predetermined distance.

7. The apparatus according to claim 5, wherein each wicking strand is able to move longitudinally in the evaporation reservoir and is at least partially buoyant in the fluid such that when fluid is present in the evaporation reservoir an end of each wicking strand is spaced from the outlet of the microfluidic channel and the fluid is able to flow out of the microfluidic channel but when the fluid level in the evaporation reservoir drops each wicking strand drops and can reach a point where each wicking strand at least partially blocks the outlet of the microfluidic channel.

8. The apparatus according to claim 1, wherein the fluid is a Newtonian fluid.

9. The apparatus according to claim 1, wherein the microfluidic channel is serpentine in plan view.

10. The apparatus according to claim 1, wherein the fluid reservoir is part of the microfluidic device.

11. The apparatus according to claim 1, wherein the evaporation reservoir is part of the microfluidic device.

12. The apparatus according to claim 1, wherein the wetting, wicking or hydrophilic structure is positioned at least partly within the evaporation reservoir such that one end of the structure is in contact with fluid at the outlet of the microfluidic channel.

13. The apparatus according to claim 1, further comprising a dead volume reservoir positioned between the microfluidic channel and the evaporation reservoir.

14. The apparatus according to claim 1, wherein the microfluidic device is relatively optically transparent.

15. The apparatus according to claim 1, wherein the microfluidic device comprises more than one microfluidic channel.

16. The apparatus according to claim 15, wherein each microfluidic claim 1, channel is driven by separate wetting, wicking or hydrophilic structures.

17. The apparatus according to when used to culture human intestinal epithelial Caco-2 cells.

18. The apparatus according to claim 1, when used to culture human lung epithelial A549 cells.

19. An arrangement comprising a plurality of the apparatus according to claim 1.

20. A method for providing fluid flow to one or more microfluidic devices, the method comprising the steps of:
providing an apparatus according to claim 1;
adding a fluid to the fluid reservoir; and
maintaining the apparatus under conditions that result in evaporation of the fluid at the outlet and create a flow of the fluid in the microfluidic channel.
